(19) United States
(12) Patent Application Publication
(10) Pub. No.: US 2004/0126318 A1
(43) Pub. Date: Jul. 1, 2004

(54) Methods, formulations and kits for monitoring and diagnosing gastric emptying and gastroparesis, and formulations for determining gastrointestinal motility

(76) Inventor: Eli D. Ehrenpreis, Skokie, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/661,948

(22) Filed: Sep. 12, 2003

Related U.S. Application Data
(60) Provisional application No. 60/410,113, filed on Sep. 12, 2002.

(51) Int. Cl. A61K 49/00; A61K 31/545; A61K 31/522

(52) U.S. Cl. 424/9.1; 424/468; 514/8; 514/23; 514/200; 514/263.34; 514/474

(57) ABSTRACT

The invention relates to methods and compositions for measuring the rate of gastric emptying in animals, including humans. The methods of the invention measure the rate of stomach emptying by administration of a marker agent in a delayed-release formulation or food vehicle and blood or serum levels of the marker agent are monitored over time. The blood or serum levels of a marker agent reflect the gastric emptying rate. Incorporating such agents in foods also delays the absorption of such agents and serves as a measure of digestion and gastric emptying. These methods may be used to screen for drugs, herbal substances and other compounds that modulate gastric emptying, such as by increasing prokinetic activity of the gastrointestinal tract.

### CROSS-REFERENCE TO RELATED APPLICATION

The priority benefit of U.S. provisional patent application serial No. 60/410,113, filed Sep. 12, 2002, the entire disclosure of which is incorporated herein by reference, is claimed.

### BACKGROUND OF THE INVENTION

1. Field of the Invention

The invention is directed to methods and compositions for monitoring gastrointestinal tract function. More particularly, the invention relates to methods and compositions for monitoring gastric emptying and gastroparesis.

2. Description of Related Technology

The primary function of the alimentary canal or gastrointestinal (GI) tract is to provide the body with a balanced supply of water, electrolytes and nutrients. In order for this to be achieved, food must be moved along the GI tract at an appropriate rate for digestion, absorption and secretion to take place. Food is normally transported through the GI tract in a well-coordinated manner by propulsive movements that are mediated by clusters of smooth muscle contractions in a process commonly referred to as peristalsis.

Gastroparesis, or delayed gastric emptying, occurs when damage to the nerves and muscles of the stomach lead to poor digestion and delayed emptying of food from the stomach to the intestines. The condition is a common complication of diabetes. Other associated conditions include scleroderma (a rheumatologic condition), progressive muscular dystrophy, amyloidosis, infection by Trypanosomes or Epstein-Barr virus, previous stomach surgery, vagotomy (or surgical cutting of the vagus nerve), visceral neuropathy, and use of a variety of medications such as anticholinergics and antidepressants. The symptoms of gastroparesis include abdominal discomfort, nausea, vomiting, malnutrition, weight loss, dehydration, bloating, early satiety, reflux, and anorexia. Patients with gastroparesis often choose not to eat since it will only make them sick. Diabetes mellitus affects about 8% of the population of the United States and most patients exhibit delayed gastric emptying after having the condition for more than ten years. The disease can also affect certain animals such as cats and horses.

The diagnosis of gastroparesis is accomplished by performing a gastric emptying scan (GES). (This test is described in detail in the Society for Nuclear Medicine Procedure Guideline for Gastric Emptying and Motility.) The GES is performed by having a subject ingest a meal containing radioactive compounds technetium-99 m (0.2 mCi-0.4 mCi) or indium-111 (0.1 mCi-0.2 mCi), either as individual compositions or together. The patient is then positioned next to, or under, a gamma camera for 90 min-240 min. The gamma camera measures radioactive counts in the stomach over time. If there is a large amount of radioactivity remaining in the stomach at the end of the test period, then the emptying rate of the stomach is slow and gastroparesis is diagnosed. The test also has been used to calculate the gastric emptying rate and lag time of gastric contents prior to emptying. These parameters can also be used to diagnose gastric emptying disorders.

Blockage of the stomach or small bowel can produce the same symptoms as gastroparesis. Therefore, an endoscopy or x-ray study often is performed, either initially or after the GES test in order to rule out the possibility that the slow gastric emptying is a result of physical blockage of the emptying route. Thus, a diagnosis of gastroparesis is made after obstructive causes of delayed gastric emptying, such as tumors or strictures have been eliminated. Antral-duodenal manometry (measurement of the strength and timing of stomach contractions) or an electrogastrogram (measurement of the electrical activity of the stomach, like an EKG) may also be performed. Despite the fact that the GES provides a diagnosis of gastroparesis, it has disadvantages in that it is costly and time consuming. Additionally, GES exposes subjects to radioactivity (the largest dose to an organ ranges from 0.11 mGy to 2 mGy) and generates radioactive and toxic wastes and requires performance of the test by a nuclear medicine technician. Further, a physician specialist is needed to interpret results of the study.

The “flapjack test” is another test that has been employed to monitor gastric emptying. This test uses a [¹³C]1-sodium acetate labeled “flapjack” consumed by the patient and monitored using a breath test. It is an extension of the [¹³C]1-octanoic acid breath test, which whisks the labeled octanoic acid into egg yolks prior to making an omelet. While this test has advantages over the traditional test, for example, it is applicable to children and is suitable for office use, it requires an isotope of carbon to perform as well as a device to measure isotope concentrations and is therefore costly.

Another test for gastric emptying is administration of polysterene beads, such as STYROFOAM™ beads via gavage to fasted rats. The movement of these beads out of the stomach is monitored. The rats are sacrificed over varying time periods in order to determine the content of the beads in the stomach and intestine. This test is undesirable because it is expensive, requires sacrifice of the animals and does not account for the effect that STYROFOAM™ beads may have on gastric motility.

The prokinetic assay in rats is a well-established model for identifying compounds that possess prokinetic activity (e.g., Dropleman et al., J., Pharmacol. Methods, 4(3):227-30, 1980). In this model, a test meal is prepared by slowly adding cellulose gum to cold distilled water. Beef bouillon cubes are dissolved in warm water and then blended into the cellulose solution followed by purified casein, powdered confectioner's sugar, cornstarch and powdered charcoal. Each ingredient is added slowly and mixed thoroughly, resulting in a dark gray to black, homogenous paste. The meal is then refrigerated overnight during which time trapped air escapes. Prior to the assay the meal is removed from the refrigerator and allowed to warm to room temperature. Mature rats are deprived of food for 24 hours. On the morning of the study, each animal is weighed and randomly assigned to treatment groups e.g., ten animals per group.

Each rat receives either vehicle, test compound or the reference standard metoclopramide by intraperitoneal injection. At 0.5 hours post-injection, the test meal is orally administered to each rat with a syringe. At 1.5 hours post-injection each rat is sacrificed by carbon dioxide asphyxiation and the stomach is removed by opening the abdomen and carefully clamping and cutting the esophagus just below the pyloric sphincter. Taking care not to lose any of the contents, each stomach is immediately weighed. Each stomach is then cut open along the lesser curvature, rinsed with tap water, gently blotted dry to remove excess moisture and weighed again. The amount of test meal remaining in the stomach is represented by the difference between the weight of the full stomach and the weight of the stomach empty. The difference between the amount of the test meal remaining and the mean test meal weight represents the quantity of test meal that empties during the 1.5 hour post-injection period.

Another prokinetic assay uses dogs with several strain-gauge transducers sutured to various tissues to monitor contractile activity of the circular muscle layer of the stomach or intestine. The prokinetic agent is added post-surgically to the animal and variations in gastric motility are measured using the strain gauges.

The prokinetic tests described above are clearly time consuming, require surgery and/or sacrifice of the test animal and are expensive to conduct. Additionally, prokinetic properties determined in laboratory animals may not directly correlate with these drug effects in humans. Thus there is a need for a relatively inexpensive, non-invasive and reproducible technique for measuring the rate of gastric emptying in animals, including humans, to substitute for conventional gastric emptying tests.

In addition to difficulties in diagnosing gastroparesis, this disorder is one of the most difficult gastrointestinal disorders to treat. This condition occurs in patients with a marked reduction in the rate that materials empty from the stomach. Delayed gastric emptying also plays a role in non-ulcer dyspepsia, another difficult-to-manage functional gastrointestinal disorder. The only medications currently used to treat gastroparesis and delayed gastric emptying are cholinergic agents, cisapride (Propulsid), erythromycin and metochlopromide (Reglan).

These drugs provide some benefit to those who take them, but they have a number of disadvantages. The FDA removed cisapride from the market because it produced severe and sometimes fatal arrhythmias. Oral erythromycin has not been proven to be effective for treatment of gastroparesis. Metochlopramide has a number of serious side effects including drowsiness, nightmares, depression and Parkinsonism that occur in up to 50% of patients treated with this drug. Cholinergic drugs, like bethanechol, are associated with serious and sometimes life-threatening side effects such as cardiovascular collapse and cardiac arrhythmias. Some patients with gastroparesis have to rely on nutritional support using an intestinal feeding tube to keep them alive. This results in a marked decrease in the patient's quality of life. Implantation of electrodes into the stomach to stimulate contractions is a new experimental technique being developed as a treatment for gastroparesis. For patients that are refractory to all available treatments, a gastrectomy (or surgical removal of the stomach) is performed. At present, there is no cure for gastroparesis.

Thus, there is a need to develop methods and compositions that may be used for diagnosing gastroparesis and/or other gastric emptying disorders and concomitantly there also exists a need for identifying new agents (prokinetic agents) for the treatment of gastric emptying disorders. Such disorders, including but not limited to, delayed gastric emptying, diabetic gastroparesis, non-ulcer dyspepsia, anorexia, gallbladder stasis, surgically induced adynamic ileus and chronic constipation may all benefit from treatment with prokinetic agents. In addition, prokinetic agents can aid in the placement of diagnostic and therapeutic instrumentation, such as during the insertion of enteral feeding tubes into the proximal small intestine and when performing small and large intestinal imaging studies.

### SUMMARY OF THE INVENTION

The invention is directed generally to methods and compositions for monitoring gastrointestinal tract function. In particular embodiments, the invention provides methods of monitoring gastric emptying in a mammal comprising administering to the mammal a formulation comprising an agent that is formulated in a delayed-release formulation that prevents the agent from being released into the gastrointestinal tract when the pH of the gastrointestinal tract is lower than about 6.0, and determining the amount of time taken for an elevated concentration of the agent to be found in the blood of the mammal. Preferably, the amount of time taken for elevated concentrations to be found in the blood of the mammal is greater than five minutes. The agent being released into the gastrointestinal tract is preferably a substance not present in normal dietary substances, thereby facilitiating ease of detection of the substance from other components of the gastric milieu.

In particular embodiments, the agent is a D-sugar selected from the group consisting of D-xylose, D-galactose, D-mannose, D-fructose, L-fucose, L-rhamnose and L-sorbose. Alternatively, the agent may be selected from the group consisting of acetominophen, aspirin, caffeine, cephalosporins, beta-lactam antibiotics, cimetidine, ranitidine, famotidine, nizaditine, alprazolam, gentamicin, amikacin, vancomycin, diclofenac, ibuprofen, D-amino acids, beta carotene, ascorbic acid, sulfur dioxide, biotin, inositol, zinc, vitamin B12, folate, aluminum sulfate, eugenol, citral, vanillin, and malic acid.

Regardless of the identity of the agent, it is preferably encapsulated in a pH-sensitive formulation. Certain embodiments contemplate that the agent is non-isotopic. In particular embodiments the agent is formaulated as part of a test meal or drink.

The invention further contemplates methods of diagnosing a gastric emptying disorder in a mammal comprising administering to the mammal a formulation comprising an agent that is not released into the gastrointestinal tract at a pH lower than about 6.0, and determining the amount of time taken, post-administration, for an elevated concentration of the agent to be found in the blood of the mammal, wherein the mammal is diagnosed as having a gastric emptying disorder if the agent is not elevated in the blood stream 120 minutes post-administration.

Another aspect of the invention is directed to a method of diagnosing gastroparesis in a human comprising administering to the human a formulation comprising D-xylose that is not released into the gastrointestinal tract at a pH lower than about 6.0, and determining the amount of time taken, post-administration, for an elevated concentration of the D-xylose to be found in the blood of the human, wherein the human is diagnosed as having a gastric emptying disorder if the agent is not elevated in the bloodstream 120 minutes post-administration, wherein the human has previously been or is later determined not to have a blockage of the stomach or small bowel.

In the diagnostic methods of the invention, the total dosage of D-xylose administered is preferably between about 5 grams and about 25 grams.

Also contemplated herein is a method of screening for a compound that modulates gastrointestinal motility in a mammal comprising administering a test compound to the mammal; monitoring the effect of the test compound on gastric emptying by administering to the mammal a formulation comprising an agent that is not released into the gastrointestinal tract at a pH lower than about 6.0, and determining the amount of time taken, post-administration, for an elevated concentration of the agent to be found in the blood of the mammal in the presence and absence of the test compound, wherein a test compound which alters the rate of gastric emptying is identified as a modulator of gastrointestinal motility. In specific embodiments, it is contemplated that the test compound increases gastrointestinal motility as determined by an increase in the time from administration to appearance of the agent in the blood of the mammal. In certain embodiments, the test compound decreases gastrointestinal motility as determined by a decrease in the time from administration to appearance of the agent in the blood of the mammal.

Also contemplated to be within the bounds of the invention are modulators identified according to the screening assays of the invention. Preferably, such modulators are identified as prokinetic agents.

The invention further encompasses a formulation for determining gastrointestinal motility comprising between about 250 mg and 1000 mg of D-xylose and a pharmaceutically acceptable carrier, diluent, or excipient. In specific embodiments, such a formulation is prepared as a test meal.

Also provided herein is a kit for monitoring gastric emptying, comprising a first marker agent formulated in a delayed-release formulation that prevents the first marker agent from being released at a pH lower than about 6.0, and instructions for using the formulation for determining gastric emptying. Such kits may preferably comprise a formulation comprising between about 250 mg and 1000 mg of D-xylose and a pharmaceutically acceptable carrier, diluent, or excipient and instructions for the performance of an assay for determining gastric motility. The kits may further comprise a blood collection device. Alternatively, or further, the kit may also comprise a composition for detecting the presence of the marker agent.

In certain embodiments, any or all of the above kits may further comprise a second marker agent formulated in a formulation that prevents the second marker agent from being released at a pH lower than about 6.0. Such a second marker agent may be formulated in the same delayed-release formulation as the first marker agent.

In certain embodiments, any or all of the kits may also comprise a series of concentrations of marker agent in delayed release formulations for use in producing a standard curve, the formulations each being resistant to release of the marker agent at a pH lower than about 6.0. In certain other embodiments, any or all of the aforementioned kits also may further comprise a reagent that lowers the pH of the formulation to a pH lower than about 6.0.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE FIGURES

FIG. 1 shows a graph of serum D-xylose concentration as a function of time.

FIG. 2 shows a curve of serum D-xylose as a function of time curve after ingestion of D-xylose in water versus ingestion of D-xylose incorporated into a brownie.

### DETAILED DESCRIPTION

Defects in the normal motility pattern of the GI tract can lead to the development of chronic, painful and debilitating disorders. For example, an incompetent or weak lower esophageal sphincter may result in frequent reflux of ingested food from the stomach into the esophagus, which may lead to esophagitis and ultimately esophageal ulcers. Prokinetic agents (also called motility-enhancing agents) are useful in treating reflux esophagitis because they (a) increase the pressure of the lower esophageal sphincter, thereby inhibiting reflux; (b) increase the force of esophageal peristalsis to facilitate clearance of food from the esophagus into the stomach; and (c) increase gastric emptying, thereby further decreasing the mass available for reflux.

Thus, the invention provides an inexpensive, non-invasive and reproducible technique for measuring the rate of gastric emptying in animals, including humans. The technique is a substitute for conventional gastric emptying tests to diagnose delayed gastric emptying without using radioactive materials. Furthermore, the technique is useful in determining the prokinetic properties of drugs and other substances.

The new technique overcomes these problems by administering commercially available, nonradiographic substances that are generally regarded as safe and can be detected using a simple blood test. Further, the methods and compositions of the invention can be used in the diagnosis of gastroparesis and/or other gastric emptying disorders and also to identify new prokinetic agents for the treatment of such gastric emptying disorders, including disorders such as delayed gastric emptying, diabetic gastroparesis, non-ulcer dyspepsia, anorexia, gallbladder stasis, surgically induced adynamic ileus and chronic constipation may all benefit from treatment with prokinetic agents. The methods and compositions of the invention can further be used to evaluate the placement of diagnostic and therapeutic instrumentation, such as during the insertion of enteral feeding tubes into the proximal small intestine and when performing small and large intestinal imaging studies.

In a preferred embodiment, D-xylose, a five-carbon sugar not found in normal dietary substances, is administered to a patient in a delayed release capsule (such as EUDRAGIT-L® capsules) and blood levels of D-xylose are measured over several time points. Because D-xylose in EUDRAGIT-L® capsules is only released when the capsules are dissolved, which requires a pH of 6 or greater, the substance only appears in the bloodstream after the capsules are emptied from the stomach. D-xylose or other substances can be utilized in this manner using a variety of capsules that have release properties dependent on pH, capsule size or digestion by intestinal secretions. Measurements of D-xylose or other substances in the blood or serum (particularly those that are rapidly absorbed after release) is therefore an indirect measure of gastric emptying and capsule dissolution. Measured serum levels of D-xylose can then be used to estimate the gastric emptying rate and to diagnose delayed gastric emptying.

In an alternate use of the test, the prokinetic properties of drugs and other therapeutic molecules can be determined in humans, again without the need for radioactive materials and nuclear specialists to evaluate the data. This is accomplished by performing repeated testing with capsule ingestion before and after administration of the prokinetic agent. The blood or serum curves generated from these studies may be used to evaluate the effect of the agent on gastric emptying.

The term “prokinetic” means the enhancement of peristalsis in, and thus movement through, the gastrointestinal tract.

The term “gastroparesis” means a partial or complete paralysis of the stomach brought about by a motor abnormality in the stomach or as a complication of diseases such as diabetes, progressive systemic sclerosis, anorexia nervosa, or myotonic dystrophy.

By “acidic pH,” the invention means any pH that is lower than about pH 6.0.

A “pH-sensitive” formulation is one which is sensitive to the pH of the surrounding environment. In the context of the invention, the pH-sensitive formulation is one which is resistant to release of the active agents contained in the formulation at a pH lower than about 6.0.

A “marker agent” in the invention is any agent whose path through the gastrointestinal tract can be monitored.

A “gastric mobility modulator” in the invention is any agent which alters the gastric mobility either by decreasing or increasing gastric mobility.

The invention provides methods and compositions for the diagnosis and treatment of gastrointestinal disorders. More specifically, the invention uses delayed release formulations of marker/tracer agents that are administered to an animal and determines the presence of the marker in the blood stream of the animal. The delayed release formulation is one which does not release the marker agent into the gastrointestinal tract until the formulation has emptied from the stomach. One preferred method of achieving such a formulation is to prepare the delayed release formulation such that the marker agent, e.g., a D-sugar or another agent that is not normally found in the bloodstream of an animal, is sequestered in a carrier and not released at a pH lower than about 6.0. Since the pH of the stomach is acidic and lower than about pH 6.0, the marker agent will not be released and taken up into the blood of the animal until the formulation has emptied from the stomach.

The invention contemplates the use of any formulation which does not allow the release of the marker agent in the mouth, esophagus or stomach but allows the release of the agent once the formulation has passed through the stomach into the small intestine and other post-stomach regions of the gastrointestinal tract. The invention contemplates embedding the marker agent in a matrix which is resistant to acid pH but rapidly releases the agent in an alkaline pH. Alternatively, the marker agent is one which is presented in a particulate form in which the particles are covered by a coating that can only be removed or dissolved in non-acidic conditions. In any of the embodiments of the invention, the marker agent may be covered by a polymeric agent that is resistant to acidic pH.

Any agent that can be taken up into, and is detectable from, the bloodstream and is compatible with release in an environment of pH of about 6.0 or greater may be used as a marker agent for the invention. Such agents include, but are not limited, to fat soluble vitamins, e.g., any of vitamins A, D, E, and K, histamine antagonists, e.g., ranitidine, cimetidine and agents derived therefrom, analgesics, e.g., acetaminophen, aspirin, and the like. In particularly preferred embodiments, the marker agent is a sugar that is not naturally found in the diet of the animal. Only ten monosaccharides are found in the oligosaccharide groups of glycoconjugates in higher eukaryotic organisms: D-glucose, D-galactose, D-mannose, N-acetylglucosamine, N-acetylgalactosamine, sialic acid, glucuronic acid, iduronic acid fucose and xylose. All of these monosaccharides are of the D-configuration except fucose and iduronic acid, which have the L-configuration. D-xylose is a preferred marker in the invention as it is not naturally found in the diet of an animal such as a mammal and is normally not present in the bloodstream. However, a non-natural sugar, e.g., any sugar in the L-configuration, is useful as a marker in the invention. Such sugars can be readily detected in the blood of a mammal by performing routine chromatographic tests (e.g., see Handbook of Biochemistry and Molecular Biology: Lipids, Carbohydrates, Steroids, 3^{rd} Edition, Ed. G. D. Fasman, CRC Press, Inc, 1975).

More generally, any agent that can be conveniently detected from the blood of a subject may be used as a marker. For example, the sugars that may be used as markers in the invention include, but are not limited to, D-xylose, D-galactose, D-mannose, D-fructose. L-fucose, L-rhamnose, and L-sorbose. Pharmaceutical markers that could be used in the invention include, but are not limited to, aspirin, acetaminophen, cephalosporins (cephazolin, cefaclor, cefoperazone, etc.) penicillin, or any safe drug that is readily absorbable, measurable in the serum, and does not affect gastric motility including cismetidine, ranitidine, famotidine, nizaditine, alprazolam, gentamicin, amikacin, vancomycin, diclofenac and ibuprofen. Other compounds that could be used as markers herein include, but are not limited to, D-amino acids, beta carotene, ascorbic acid, sulfur dioxide, biotin, inositol, zinc, vitamin B12, folate, aluminum sulfate, eugenol, citral, vanillin, and malic acid.

The marker agents are formulated into pH-sensitive formulations such that the agent is only released at pH greater than about 6.0, i.e., the marker agent is not released until the formulation has passed through the stomach. For example, the marker agent may be encapsulated into a pH-sensitive microsphere, e.g., a microsphere made of polymethacrylic acid and polyethylene glycol. The pores formed by the polymeric components of such microspheres shrink in the stomach (i.e., at low pH), thereby preventing the release of the encapsulated active agent. Once the microspheres pass into the small intestine, where the pH tends towards neutral (i.e., about pH 6.0 and higher), the pores of the microspheres swell, thereby releasing the entrapped marker agent. The swelling/shrinking phenomenon is referred to as complexation (Lowman and Peppas, Macromolecules, 30 (1997) 4959-4965). Once the marker agent is released, it is readily absorbed into the blood stream. Monitoring the levels of the marker agent at various post-ingestion time intervals will allow a determination of the rate of gastric emptying in the individual.

The preparation of pH-sensitive microparticles is known to those of skill in the art (Lowman et al., In: Tailored Polymeric Materials for Controlled Delivery Systems. I. McCullough and S. Shalaby, (Eds.), ACS, Washington, D.C., ACS Symposium Series, 709, 1998, pp. 156-164, 1998). In exemplary embodiments, the marker agent is dissolved in an appropriate solvent, e.g., ethanol or ethanol solutions of various concentration in which the pH of the solution is alkaline. Dry copolymer microparticles are dispersed in a solution of the desired marker agent and stirred at a constant rate for one day. The alkaline solution causes the microparticles to swell and the marker agent is taken up into the pores of the microparticles. The weight ratio of marker agent to polymer in the initial solution may be varied e.g., from 1:1 to 1:6. Following marker loading, the solutions are filtered using 1 mm paper and an equal volume of an acid fluid is added to deswell the marker-loaded particles. These hydrogels containing the marker of interest are then dried in vacuo for 3 days and stored at 4° C. prior to use.

In alternative embodiments, the marker agent may be formulated such that it is coated with an enteric coating which is resistant to dissolution in acidic conditions. Preferably, the coating is such that the enteric coating is predisposed to dissolution in the middle and distal portions of the small intestine (see U.S. Pat. No. 5,795,882, incorporated herein by reference).

In diagnostic aspects of the invention, gastric emptying is monitored by determining the level of the marker agent in the bloodstream of the subject (human or other animal). The subject ingests the marker in a convenient edible form, e.g., a cookie, flapjack, drink, cereal or other meal substance prepared using a known amount of the marker substance. The subject will generally ingest the marker over, for example, a five to ten minute span. The blood of the subject is drawn at appropriate time intervals, e.g., every five minutes. In order to facilitate the drawing of blood at regular intervals, an intravenous catheter or other appropriate vessel may be employed over the period of the study. The period during which data is collected may vary. Data are collected for at least 60 minutes. More preferably, data are collected for at least 120 minutes and a concentration versus time curve is generated. The collected blood is placed in appropriate vessels, e.g., vacutainer tubes containing potassium oxalate and sodium fluoride (Becton Dickinson Vacutainer Systems, Rutherford, N.J., USA). The number of samples required to document the presence of gastroparesis or to demonstrate a prokinetic effect can be as few as two (Tougas et al., American Journal of Gastroenterology, Vol 95, No. 6, 2000, using a radionuclide assay). It is contemplated that 2, 3, 4, 5, 6, 7, 8, 9, 10, 15 or more samples may be taken to document the presence of gastroparesis.

Analysis of the presence of the marker agent in the blood of the subject is determined using any assay which specifically detects the marker agent in the blood. A standard concentration vs. time curve of the marker can be prepared for any such assay to facilitate a determination of the amount of marker agent appearing in the blood as compared to the amount initially ingested. Using such determinations, the half-emptying time of the subject can be determined and compared with similar measurements obtained from normal individuals who do not exhibit signs of gastroparesis or other gastric emptying disorders. From such comparisons, it is possible to determine whether the subject has a decreased (or increased) rate of gastric emptying as compared to the normal control.

The invention also contemplates the use of the methods of the invention in screening for compounds that modulate (increase or decrease activity) gastric emptying. Generally, these assays may be initially tested using laboratory test animals (e.g., mice, rats, rabbits, guinea pigs and the like), but at the clinical stage, the gastric emptying effects of the compounds may be tested on other mammals, e.g., horses, sheep, pigs, and humans.

A recent effort to simplify the diagnosis of gastroparesis has been proposed by an international group of experts in the field of gastroenterology (Tougas et al, 2000). The study demonstrated that performing a gastric emptying scan and checking a single radionuclide count four hours after administration of the label was just as efficient at yielding a diagnosis of gastroparesis as performing the entire scan. If greater than 10% of the administered dose was present after four hours, delayed gastric emptying was diagnosed. A similar diagnosis of gastroparesis is contemplated using the methods of the invention. In this aspect of the invention, the diagnostic test using D-xylose or any other marker agent uses samples drawn prior to, and at, a single time point after administration of the test substance. This time point may be any time point selected by the physician and may be for example, 10 minutes, 20 minutes, 30 minutes, 40 minutes, 50 minutes, 60 minutes, 75 minutes, 90 minutes, 105 minutes, 120 minutes, 135 minutes, 150 minutes, 180 minutes, 210 minutes, 240 minutes, 300 minutes or 360 minutes after administration of the marker compound. If the serum of the individual contains an amount of the marker compound greater than baseline levels at the selected time point, gastroparesis will be diagnosed. The baseline levels generally would be the standard levels of the marker substance in the absence of administration of the marker agent. Such baseline levels may be determined by measuring the amount or level of the marker substance at a selected time point prior to administration of the composition containing the marker agent. Alternatively, the baseline levels may be those levels of the marker agent determined to be the “average” level for a given population of subjects. The concentration of marker agent above baseline can be 5% of the total dose initially administered, 10%, 15%, 20%, 25%, 30%, 35%, 40%, 45%, 50%, 55%, 60%, 65%, 70%, 75%, 80%, 85%, 90%, 95% or 100% of the original dose that was administered.

In the screening aspects of the invention, modulators of gastric emptying are identified by monitoring gastric emptying using the methods of the invention in the presence and absence of the candidate modulator and comparing the results. It is contemplated that this screening technique will prove useful in the general identification of a compound that will serve the purpose of promoting, augmenting or increasing the therapeutic intervention of gastric emptying disorders. Such compounds are useful in the treatment of various disorders which manifest in aberrant patterns of gastric emptying, including but not limited to, dyspepsia, gastroparesis, constipation, and intestinal pseudo-obstruction, and other disorders of the gastrointestinal tract.

In certain aspects of the invention, the methods for determining the ability of a candidate substance to modulate gastric emptying generally includes the steps of

a. administering a test compound to the mammal;

b. monitoring the effects of the test compound on gastric emptying by administering to the mammal a formulation comprising an agent that is not released into the gastrointestinal tract at a pH lower than about 6.0, and

c. determining the amount of time taken for elevated concentrations of the agent to be found in the blood of the mammal in the presence and absence of the test compound, wherein a test compound which alters the rate of gastric emptying is identified as a modulator of gastrointestinal motility.

To identify a candidate substance as being capable of stimulating gastric emptying in the assay above, one would measure or determine the rate of gastric emptying before the administration of the candidate substance. One would then administer the candidate substance to the animal and determine the rate of gastric emptying in the presence of the candidate substance. A candidate substance which increases the rate of gastric emptying relative to that observed in its absence is indicative of a candidate substance being a prokinetic agent with an ability to stimulate or increase the rate of gastric emptying. Conversely, the candidate substance may be identified as one which slows the rate of gastric emptying. Such compounds may be useful in the treatment or amelioration of disorders which manifest diarrhea or too rapid a rate of gastric emptying.

As used herein the term “candidate substance” refers to any molecule that is capable of modulating or otherwise altering the gastric motility. In specific embodiments, the candidate substance molecule is one which modulates gastroparesis. The candidate substance may be a protein or fragment thereof, a small molecule, or even a nucleic acid molecule. The most useful pharmacological compounds for identification through application of the screening assay may be compounds that are structurally related to other known modulators of gastric emptying. In this regard, the candidate substance may be one that is structurally related to existing prokinetic agents such as cisapride (see U.S. Pat. No. 5,955,477), norcisapride (see U.S. Pat. Nos. 6,353,005 and 6,362,202), macrocyclic lactam derivatives of erythromycins A, B, C and D (see U.S. Pat. Nos. 5,554,605; 5,538,961; 5,523,418; and 5,523,401), and 3-hydroxy-piperidinemethanol derivatives (see U.S. Pat. No. 6,096,761) (these patents are incorporated herein by reference in their entirety and provide additional background information relating to the disorders that may be diagnosed by the invention).

In addition, approved drugs that are already on the pharmaceutical market may also be tested for effects on gastric motility. Certain approved drugs with potential prokinetic activities that have previously not been tested for such activity include yohimbine, guafenesin, orlistat, prostaglandin analogs, donepezile, selective solution receptor uptake agents, venlafaxine, dextromethorphan, methylnaltrexone, naloxone, naltrexone and others known in the art. Potential herbal agents to be tested include cat's claw, devil's claw, cayenne, mint, ginger and others known in the art.

The active compounds may include fragments or parts of naturally-occurring compounds or may be found only as active combinations of known compounds which are otherwise inactive. The active compounds may include fragments or parts of naturally occurring compounds or may be found as active combinations of known compounds which are otherwise inactive. Accordingly, the invention provides screening assays to identify agents which stimulate or inhibit gastric emptying. It is proposed that compounds isolated from natural sources, such as animals, bacteria, fungi, plant sources (including leaves and bark), and marine samples may be assayed as candidates for the presence of potentially useful pharmaceutical agents.

The pharmaceutical agents to be screened could also be derived or synthesized from chemical compositions or man-made compounds. Thus, it is understood that the candidate substance identified by the invention may be polypeptide, polynucleotide, small molecules or any other inorganic or organic chemical compounds that may be designed through rational drug design starting from known stimulators or inhibitors of gastric emptying.

The candidate screening assays are simple to set up and perform. Thus, in assaying for a candidate substance, one would initially determine the rate of gastric emptying in an animal prior to the administration of the test compound. Subsequently, the test compound is administered and the rate of gastric emptying is re-determined. In this fashion, one can measure the ability of the candidate substance to modulate gastric emptying in the animal. Treatment of animals with test compounds will involve the administration of the compound, in an appropriate form, to the animal. Administration is achieved by any route that can be utilized for clinical or non-clinical purposes, including, but not limited to, oral, nasal, buccal, rectal, vaginal or topical. Alternatively, administration may be by intratracheal instillation, bronchial instillation, intradermal, subcutaneous, intramuscular, intraperitoneal or intravenous injection.

“Effective amounts” are those amounts effective to reproducibly alter the rate of gastric emptying in an animal as compared to their normal levels. Compounds that achieve significant appropriate changes in activity are preferred. Such compounds also may be compared with known modulators of gastric emptying.

Significant changes in the rate of gastric emptying as observed using the gastric emptying test described in the present application are represented by an increase or decrease in activity of at least about 20%-40%, and, most preferably, by changes of at least about 50%, with higher values also contemplated.

There are a number of different libraries used for the identification of small molecule modulators that affect gastric emptying including chemical libraries, natural product libraries, and combinatorial libraries comprised of random or designed peptides, oligonucleotides, or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review, see Science 282:63-68 (1998). Combinatorial libraries are composed of large numbers of peptides, oligonucleotides, or organic compounds as a mixture. They are relatively simple to prepare by traditional automated synthesis methods, PCR cloning or other synthetic methods. Of particular interest will be libraries that include peptide, protein, peptidomimetic, multiparallel synthetic collections, recombinatorial and polypeptide libraries. For a review of combinatorial libraries and libraries created therefrom, see Myers, Curr. Opin. Biotechnol. 8: 701-707 (1997). A candidate modulator identified by the use of the various libraries described may then be optimized for its activity as a prokinetic or other gastric emptying modulator through, for example, rational drug design. It will, of course, be understood that all the screening methods of the invention are useful in themselves, notwithstanding the fact that effective candidates may not be found.

The goal of rational drug design is to produce structural analogs of biologically active compounds that are known to have the desired effect. By creating such analogs, it is possible to fashion drugs which are more active or stable than the natural molecules, which have different susceptibility to alteration, or which may affect the function of various other molecules. By virtue of the availability of existing prokinetic agents, sufficient amounts of the related compounds can be produced and tested using the methods of the invention.

The invention further contemplates the treatment of disease states that manifest a symptom of altered gastric emptying. Moreover, the gastric emptying assays described in the invention can be used to monitor the effectiveness of a given therapy in clinical trials, or in monitoring the treatment of an individual patient. In order to provide a basis for the diagnosis of disease, a normal or standard gastric emptying profile for the subject is established. The profile of the test subject may be compared to one obtained from normal subjects. Deviation between standard and subject values establishes the presence of an abnormality in gastric emptying.

Once an abnormality in gastric emptying is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

For use in these diagnostic and therapeutic embodiments, all of the components required for the assays may be conveniently presented in kit form. The kit may include a set of instructions for using the marker compounds and other components to carry out the method of determining gastric emptying in a subject, as described above. The instruction set provides information in any suitable format (e.g., printed on paper or in electronic format on a diskette, CD-ROM, or by reference to a web site or printed publication) to allow the user to collect a suitable specimen for determining the serum levels of the marker compound, process the specimen, use the marker compound or compounds to determine the amount of marker compound present in the bloodstream of the subject at a given time post-administration of the marker agent, and to interpret the results obtained, i.e., to compare the results to a threshold which allows the user to determine the level and/or rate of gastric emptying of the subject and to diagnose whether the subject manifests symptoms of gastroparesis.

The kits of the invention further may contain blood collection devices, such as, for example vacutainer tubes (Becton Dickinson Vacutainer Systems, Rutherford, N.J., USA). In addition, the kits may comprise all the reagents necessary to detect and determine the concentrations of a given marker agent in a specimen. Such detection reagents may be any reagent that will allow the determination of the presence of the marker agent and may include antibodies specific for the given marker agent, fluorophores, or chromaphores for detecting the marker agent, chromatographic media for detecting the marker agent (e.g., thin-layer chromatography strips on which the marker agent may be separated from the specimen collected from the subject), and components of ELISA-type assays for detecting the presence of the marker agent.

Where clinical applications are contemplated, it is desirable to prepare the marker agents and/or the prokinetic agent compositions identified by the invention as pharmaceutical compositions, i.e., in a form appropriate for in vivo applications. Generally, this will entail preparing compositions that are essentially free of pyrogens, as well as other impurities that could be harmful to humans or animals.

One will generally desire to employ appropriate salts and buffers to render the compositions stable and to allow for uptake once the marker agents have passed through the stomach. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption-delaying agents, and the like. The use of such media and agents for pharmaceutically active substances is well known in the art. Except insofar as any conventional media or agent is incompatible with the compositions used in the invention, its use in therapeutic compositions is contemplated. Supplementary active ingredients also can be incorporated into the compositions. In particular embodiments, it is contemplated that the marker agents used in the gastric emptying assays of the invention may be used to prepare a test meal. Any such meals may be used so long as the formulation is such that it does not allow the release of the marker agent at a pH of less than about 6.0.

The marker agents and/or the active compounds may be prepared for administration as solutions of a free base or pharmacologically acceptable salts thereof and/or in water suitably mixed with a surfactant, such as hydroxypropylcellulose. Dispersions also can be prepared in glycerol, liquid polyethylene glycols, in mixtures thereof, and in oils. Under ordinary conditions of storage and use, these preparations contain a preservative to prevent the growth of microorganisms. In all cases the form is preferably sterile and stable under the conditions of manufacture and storage and is preferably preserved against the contaminating action of microorganisms, such as bacteria and fungi.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the compositions.

For oral administration, the markers used in the assays of the invention may be combined with excipients. The active ingredient may also be dispersed in dentifrices, including gels, pastes, powders, and slurries.

The compositions of the invention may be formulated in a neutral or salt form. Pharmaceutically acceptable salts include the acid-addition salts of peptides (formed with the free amino groups of the protein), which are formed with inorganic acids such as, for example, hydrochloric or phosphoric acids, or such organic acids as acetic, oxalic, tartaric, mandelic, and the like. Salts formed from free carboxyl groups also can be derived from inorganic bases such as, for example, sodium, potassium, ammonium, calcium, or ferric hydroxides, and such organic bases as isopropylamine, trimethylamine, histidine, procaine, and the like.

The assays described in the invention may be useful in fields of human medicine and veterinary medicine. Thus, the subject to be treated may be a mammal, preferably human. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs, and hamsters; and poultry such as chickens, turkey, ducks and geese.

Exemplary embodiments for practicing the invention are described in the following examples.

### EXAMPLE 1

### Measurement of Gastric Emptying By Using D-Xylose in a Delayed-Release Capsule

In a preferred embodiment, gastric emptying is measured by using D-xylose in a delayed-release capsule. D-xylose, a 5-carbon sugar that is readily absorbed in the small intestine, is packaged in delayed-release capsules (such as EUDRAGIT-L®). D-xylose capsules will contain various doses, including at least 50 mg, at least 100 mg, at least 150 mg, at least 200 mg, at least 250 mg, at least 300 mg, at least 350 mg, at least 400 mg, at least 450 mg, at least 500 mg, at least 600 mg, at least 700 mg, at least 750 mg, at least 800 mg, at least 900 mg, at least 1 gram, or more than 1 gram, in different capsule sizes. The optimal capsule sizes and compound doses are determined by the results of preliminary studies and can vary with the age, size and weight of the subject such as a patient. The patient swallows one or more of the capsules with water or another liquid vehicle (e.g., saliva). When D-xylose is ingested in this form, the capsules remain intact until reaching a portion of the intestinal tract having a pH greater than about 6. Because the pH of the stomach is acidic (generally between pH 1 and 3), the capsules remain intact in the stomach. Upon entering the small intestine, where the pH is about 6.0 or greater, dissolution of the capsules occurs and the D-xylose is released and absorbed. Thus, release of D-xylose does not occur until after the capsules are emptied from the stomach, limiting its absorption into the bloodstream until after the capsules have passed through the stomach. Once the capsules are dissolved, D-xylose is rapidly absorbed. Blood is drawn from the patient at various times after administration of the capsule and examined for the presence of D-xylose.

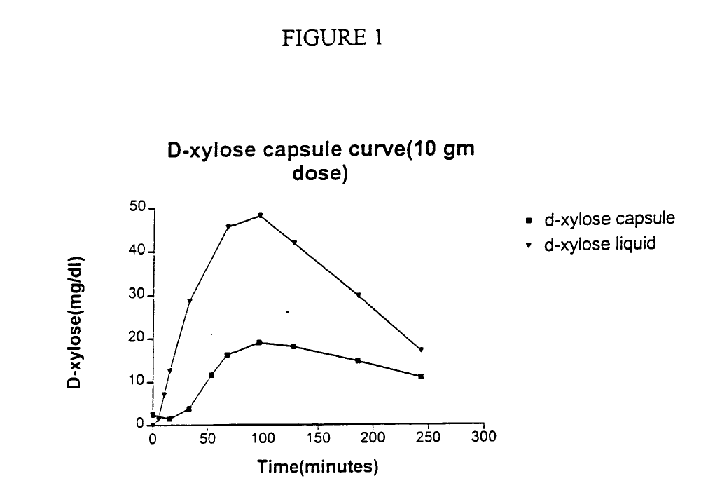

FIG. 1 shows a graph of serum D-xylose concentration as a function of time. One curve represents average data for 5 normal volunteers ingesting D-xylose mixed in water. The other curve is data obtained from a normal volunteer ingesting D-xylose contained within pH-sensitive capsules (EUDRAGIT-L®) that release D-xylose at a pH of 6 or higher. Because the stomach contains acid at a very low pH, (usually pH of 3 or less), D-xylose is only released upon entering the small intestine after being emptied from the stomach. D-xylose is normally only absorbed in the small intestine; none is absorbed in the stomach. The curve of the D-xylose concentrations from the capsule ingestion is much different from the curve for the D-xylose mixed in water. The D-xylose capsule curve shows a delay (or lag) prior to D-xylose appearance in the serum. Additionally, the slope of the initial absorption portion and terminal portions of the curve are shallower then the slope of the D-xylose in liquid curve, demonstrating slower adsorption or uptake of D-xylose due to delayed entry into the small intestine.

The delay seen in the D-xylose concentration versus time curves occurs because no D-xylose enters into the bloodstream during the time that the capsules are in the stomach. Thus, this delay or lag period, represented as a decrease in the rate of test compound (xylose, drug, etc.) uptake, is a direct reflection of the gastric emptying time. This lag time and emptying rate are determined by measurement of serum D-xylose levels after capsule ingestion. These parameters are easily reproducible, and can differ greatly between normal individuals and those with delayed gastric emptying.

### EXAMPLE 2

Measurement of Gastric Emptying by Using Acetaminophen or other Nontoxic Agents that are Rapidly Absorbed in the Small Intestine in a Delayed-Release Capsule

Acetaminophen, a commonly used analgesic and antipyretic, is rapidly absorbed from the small intestine following oral ingestion. Acetaminophen must be emptied from the stomach prior to absorption in the small intestine. Using this method, acetaminophen is packaged in delayed-release capsules (such as EUDRAGIT-L®). Various doses and capsule sizes of acetaminophen may be used, as has been described for the D-xylose in Example 1. The appropriate dose and capsule size is determined using data from preliminary studies. As previously described, when acetaminophen or any suitable nontoxic substance (such as aspirin, caffeine, and the like) are ingested in this form, the capsules pass intact through the stomach and dissolve in the small intestine. Upon dissolution of the capsule, acetaminophen, aspirin or other compounds are then released and absorbed, i.e., only after gastric emptying. Blood is again drawn from the patient according to standard medical procedures and analyzed for acetaminophen (or aspirin, caffeine, and the like) concentration.

The delay seen in the curves of acetaminophen concentration versus time, (or the concentration versus time curves for other ingested substances), as well as the decrease in absorption rates of these substances, reflects the inability of these substances to enter into the bloodstream during the time that the capsules are in the stomach. Thus, this delay or lag period, as well as the measured delay in drug uptake, is a direct reflection of the gastric emptying time. This lag time and emptying rate is reproducible and differs greatly between normal individuals and those with delayed gastric emptying.

### EXAMPLE 3

Use of D-Xylose or other Substances Incorporated into Foods

This method allows the incorporation of D-xylose or other marker agents, such as the marker agents described herein, into foods such as brownies, pancakes, cake, or other appropriate foods as the vehicle. Ingestion of these substances is followed by measurement of serum D-xylose levels at various time intervals. Because the D-xylose in this formulation is not absorbed until it enters the small intestine, the results of the D-xylose concentration vs. time curve reflect the delay in D-xylose uptake resulting from digestion of the food substance and release of D-xylose after such digestion in the stomach.

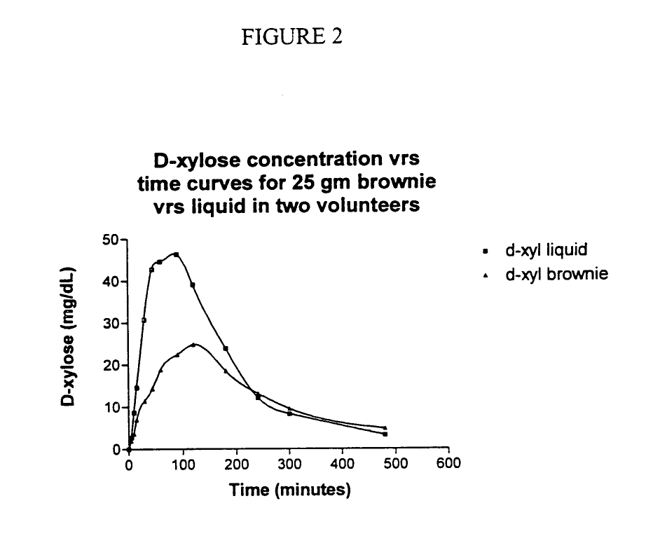

FIG. 2 shows a curve of serum D-xylose as a function of time curve after ingestion of D-xylose in water versus ingestion of D-xylose incorporated into a brownie. As with the delayed release capsules, the uptake of D-xylose is markedly delayed. This delay reflects the process of brownie digestion, release of D-xylose from the brownie, gastric emptying and absorption of the released D-xylose from the small intestine. This type of measurement provides a physiological evaluation of digestion of food substances, primarily occurring in the stomach, as well as gastric emptying. Other substances such as acetaminophen, aspirin, caffeine, and the like are measured in a similar fashion.

### EXAMPLE 4

Measurement of the Prokinetic Properties of Drugs

The invention provides assays that are inexpensive, noninvasive. These assays do not result in exposure to toxic or radioactive compounds. Further these assays are easily reproduced in the same individual before and after ingestion of a drug or other substance. Using one of the three examples described above, the marker agent is administered to the patient prior to (for baseline purposes) and during administration or immediately after administration of a candidate drug (modulator of gastric emptying e.g., a prokinetic drug) that is given to modulate the gastric emptying. Blood is again drawn at various time points and tested for serum concentrations of the marker agent (D-xylose, acetaminophen, aspirin, caffeine, etc.). The baseline absorption rate of the marker agent is compared with the absorption rate after the candidate drug has been administered. Changes in the serum concentration vs. time curves after administration of the candidate modulator/drug can show a change, such as a shortening of the lag time and, in this circumstance a more rapid uptake of the substance by the small intestine. This then provides a simple safe, nonradiographic test to screen drugs, herbal compounds, and other candidate drug substances for modulation of gastric emptying rates, and to identify such drugs, herbal compounds or other candidate drug substances as being e.g., prokinetic agents. Agents identified in this manner may be further investigated and compared to conventional, preexisting prokinetic agents and their comparative therapeutic value may be ascertained.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all incorporated herein by reference.

What is claimed is:

1. A method of monitoring gastric emptying in a mammal comprising:
a. administering to said mammal a formulation comprising an agent that is formulated in a delayed-release formulation that prevents said agent from being released into the gastrointestinal tract when the pH of the gastrointestinal tract is lower than about 6.0, and
b. determining the amount of time taken for an elevated concentration of said agent to be found in the blood of said mammal.

2. The method of claim 1, wherein the amount of time taken for elevated concentrations to be found in the blood of said mammal is greater than five minutes.

3. The method of claim 1, wherein said agent is an agent that is not present in normal dietary substances.

4. The method of claim 1, wherein said agent is a sugar.

5. The method of claim 4, wherein said sugar is selected from the group consisting of D-xylose, D-galactose, D-mannose, D-fructose, L-fucose, L-rhamnose, and L-sorbose.

6. The method of claim 1, wherein said agent is selected from the group consisting of acetominophen, aspirin, caffeine, cephalosporins, beta-lactam antibiotics, cimetidine, ranitidine, famotidine, nizaditine, alprazolam, gentamicin, amikacin, vancomycin, diclofenac, ibuprofen, D-amino acids, beta carotene, ascorbic acid, sulfur dioxide, biotin, inositol, zinc, vitamin B12, folate, aluminum sulfate, eugenol, citral, vanillin, and malic acid.

7. The method of claim 1, wherein said agent is encapsulated in a pH-sensitive formulation.

8. The method of claim 1, wherein said agent is non-isotopic.

9. The method of claim 1, wherein said agent has been formulated into a test meal.

10. A method of diagnosing a gastric emptying disorder in a mammal comprising:
a. administering to said mammal a formulation comprising an agent that is not released into the gastrointestinal tract at a pH lower than about 6.0, and
b. determining the amount of time taken, post-administration, for an elevated concentration of said agent to be found in the blood of said mammal, wherein said mammal is diagnosed as having a gastric emptying disorder if said agent is not elevated in the blood stream 120 minutes post-administration.

11. A method of diagnosing gastroparesis in a human comprising:
a. administering to said human a formulation comprising D-xylose that is not released into the gastrointestinal tract at a pH lower than about 6.0, and
b. determining the amount of time taken, post-administration, for an elevated concentration of said D-xylose to be found in the blood of said human, wherein said human is diagnosed as having a gastric emptying disorder if said agent is not elevated in the bloodstream 120 minutes post-administration,
wherein said human has previously been or is later determined not to have a blockage of the stomach or small bowel.

12. The method of claim 11, wherein the total dosage of D-xylose administered is between about 5 grams and about 25 grams.

13. A method of screening for a compound that modulates gastrointestinal motility in a mammal comprising:
a. administering a test compound to said mammal;
b. monitoring the effect of said test compound on gastric emptying by administering to said mammal a formulation comprising an agent that is not released into the gastrointestinal tract at a pH lower than about 6.0, and
c. determining the amount of time taken, post-administration, for an elevated concentration of said agent to be found in the blood of said mammal in the presence and absence of said test compound, wherein a test compound which alters the rate of gastric emptying is identified as a modulator of gastrointestinal motility.

14. The method of claim 13, wherein said test compound increases gastrointestinal motility as determined by an increase in the time from administration to appearance of said agent in the blood of said mammal.

15. The method of claim 13, wherein said test compound decreases gastrointestinal motility as determined by a decrease in the time from administration to appearance of said agent in the blood of said mammal.

16. A modulator of gastrointestinal motility identified according to the method of claim 13.

17. The modulator of claim 16, wherein said modulator is a prokinetic agent.

18. A formulation for determining gastrointestinal motility comprising between about 250 mg and 1000 mg of D-xylose and a pharmaceutically acceptable carrier, diluent, or excipient.

19. The formulation of claim 18, wherein said formulation is prepared as a test meal.

20. A kit comprising the formulation of claim 18 and instructions for the performance of an assay for determining gastric motility.

21. The kit of claim 20, further comprising a blood collection device.

22. The kit of claim 20, further comprising a composition for detecting the presence of the marker agent.

23. The kit of claim 20, further comprising a second marker agent formulated in a formulation that prevents said second marker agent from being released at a pH lower than about 6.0.

24. The kit of claim 23, wherein said second marker agent is formulated in the same delayed-release formulation as said first marker agent.

25. The kit of claim 20, further comprising a series of concentrations of marker agent in delayed release formulations for use in producing a standard curve, said formulations each being resistant to release of said marker agent at a pH lower than about 6.0.

26. The kit of claim 25, further comprising a reagent that lowers the pH of the formulation to a pH lower than about 6.0.

27. A kit for monitoring gastric emptying, comprising a first marker agent formulated in a delayed-release formulation that prevents said first marker agent from being released at a pH lower than about 6.0, and instructions for using said formulation for determining gastric emptying.

28. The kit of claim 27, further comprising a blood collection device.

29. The kit of claim 27, further comprising a composition for detecting the presence of the marker agent.

30. The kit of claim 27, further comprising a second marker agent formulated in a formulation that prevents said second marker agent from being released at a pH lower than about 6.0.

31. The kit of claim 30, wherein said second marker agent is formulated in the same delayed-release formulation as said first marker agent.

32. The kit of claim 27, further comprising a series of concentrations of marker agent in delayed release formulations for use in producing a standard curve, said formulations each being resistant to release of said marker agent at a pH lower than about 6.0.

33. The kit of claim 32, further comprising a reagent that lowers the pH of the formulation to a pH lower than about 6.0.

34. A kit for use in a method of claim 1, said kit comprising a first marker agent formulated in a delayed-release formulation that prevents said first marker agent from being released at pH lower than about 6.0 and instructions for the performance of the assay.

35. The kit of claim 27, further comprising a blood collection device.

36. The kit of claim 27, further comprising a composition for detecting the presence of the marker agent.

37. The kit of claim 27, further comprising a second marker agent formulated in a formulation that prevents said second marker agent from being released at a pH lower than about 6.0.

38. The kit of claim 37, wherein said second marker agent is formulated in the same delayed-release formulation as said first marker agent.

39. The kit of claim 27, further comprising a series of concentrations of marker agent in delayed release formulations for use in producing a standard curve, said formulations each being resistant to release of said marker agent at a pH lower than about 6.0.

40. The kit of claim 39, further comprising a reagent that lowers the pH of the formulation to a pH lower than about 6.0.
